(19) United States
(12) Patent Application Publication
(10) Pub. No.: US 2020/0397808 A1
(43) Pub. Date: Dec. 24, 2020

(54) TREATMENT OF STEROID-RESISTANT DISEASE USING AN ANTHOCYANIN AND A CORTICOSTEROID

(71) Applicant: THE CLEVELAND CLINIC FOUNDATION, Cleveland, OH (US)

(72) Inventors: Xiaoxia Li, Cleveland, OH (US); Caini Liu, Cleveland, OH (US); Jun Qin, Cleveland, OH (US); Ouyang Zhao, Cleveland, OH (US); Junjie Zhao, Cleveland, OH (US)

(21) Appl. No.: 16/769,960

(22) PCT Filed: Nov. 30, 2018
(86) PCT No.: PCT/US2018/063198

§ 371 Date: Jun. 4, 2020

Related U.S. Application Data
(60) Provisional application No. 62/595,753, filed on Dec. 7, 2017.

(51) Int. Cl. A61K 31/7048; A61K 31/573

(52) U.S. Cl. CPC A61K 31/7048; A61K 9/0073; A61K 31/573

(57) ABSTRACT

The present invention describes a method of treating a steroid-resistant disease or condition such as severe asthma in a subject by administering a therapeutically effective amount of an anthocyanin and a corticosteroid to a subject in need thereof.

### CROSS-REFERENCE TO RELATED APPLICATIONS

This application claims priority to U.S. Provisional Patent Application Ser. No. 62/595,753, filed Dec. 7, 2017, which is incorporated herein by reference.

### BACKGROUND

Asthma is an inflammatory disease of the airway. Based on the severity of its symptoms, the disease is divided into several different subtypes, including mild, moderate, and severe asthma. While mild and moderate asthma can be effectively managed by inhaled corticosteroid (CS), the treatment of severe asthma remains challenging. A hallmark of severe asthma is steroid insensitivity or resistance. Yim R., Koumbourlis A., Paediatr Respir Rev., 13(3), 172-6 (2012). A variety of other diseases treated by steroids are also known to sometimes become steroid-resistant.

Emerging evidence suggest that a different pathogenic mechanism underlies the development of severe asthma. Unlike mild and moderate asthma, which is characterized by eosinophilic inflammation associated with Th2 cytokines (IL-4; IL-5 and IL-13), severe asthma is associated with increased circulating IL-17A levels and neutrophilia. IL-17A is an inflammatory cytokine that can promote neutrophilic inflammation. IL-17A is increasingly recognized as the culprit cytokine driving steroid resistant auto-inflammatory diseases (e.g., multiple sclerosis and psoriasis). IL-17A-mediated inflammation is insensitive to steroid treatment as neutrophils are known to respond differently to steroids. Corticosteroids have been shown to prevent neutrophil apoptosis, enhance neutrophil recruitment and may thereby even exacerbate the disease. Banuelos et al., PLoS One., 12(5):e0177884 (2017). Accordingly, there is a need for new methods of treating steroid-resistant diseases such as steroid-resistant asthma.

### SUMMARY OF THE INVENTION

The inventors have previously identified the use of a small molecule inhibitors based on cyanidin as an inhibitor of IL-17A and IL-17ARA (IL-17A receptor subunit) interaction (IC₅₀=1.68 μM). See U.S. Patent Publication No. 2016/0068502. Cyanidin effectively alleviated several IL-17A-mediated inflammatory diseases including asthma. Cyanidin-3-glucoside (3CG), an anthocyanin, also exhibited inhibitory activity (IC₅₀=6.88 μM) for IL-17A binding to IL-17ARA. The inventors found that C3G in combined use with low-dose CS [dexamethasone (DEX)] exhibited great efficacy to alleviate asthma symptoms in a steroid-resistant neutrophilic asthma model (see FIG. 1). The beneficial effect of C3G/DEX combination is greater than that of single treatment with C3G or high-dose DEX. Therefore, the inventors expect that the invention provides an effective method for treating other steroid insensitive or resistant inflammatory diseases as well.

Accordingly, the invention provides a method of treating steroid-resistant diseases and conditions by administering a composition comprising an effective dose of an anthocyanin and a corticosteroid. The inventors have determined that the combined use of an anthocyanin and a corticosteroid provide a synergistic effect, resulting in an extremely high anti-inflammatory activity. This synergy can be achieved using doses of the compounds that were ineffective, or much less effective, when administered alone.

### BRIEF DESCRIPTION OF THE FIGURES

The present invention may be more readily understood by reference to the following figures, wherein:

FIGS. 1A and 1B provide graphs showing C3G attenuates neutrophilic airway inflammation in a HDM-induced acute asthma model. A. HDM/CFA-induced neutrophilic asthma in 8-week female C57BL/6 mice (n=8 per group). B. Shown are total and differential cell counts in BAL cell count. HDM, house dust mite. Treatment groups: Control (DMSO); Low DEX (0.3 mg/kg BW, i.p.); High DEX (3 mg/kg BW, i.p.); C3G (200 μg i.p./100 μg i.n per mouse); Low DEX/C3G. DEX and C3G were delivered to mice 24 h before HDM sensitization and 1 h before HDM challenge. Mice were sacrificed after HDM challenge. i.p., intraperitoneally; i.n. intranasally.

FIGS. 2A and 2B provide graphs showing Oral C3G attenuates neutrophilic airway inflammation in a HDM-induced acute asthma model. A. HDM/CFA-induced neutrophilic asthma in 8-week female C57BL/6 mice (n=5 per group). B. Shown are total and differential cell counts in BAL cell count. HDM, house dust mite. Treatment groups: Control (DMSO); Low DEX (0.3 mg/kg BW, i.p.); Low DEX+10 mg C3G (per mouse); Low DEX+20 mg C3G (per mouse); DEX and C3G were delivered to mice 24 h before HDM sensitization and 1 h before HDM challenge. Mice were sacrificed after HDM challenge.

FIGS. 3A and 3B provide graphs showing C3G attenuates neutrophilic airway inflammation in a Th17-induced asthma model. A. HDM/CFA-induced neutrophilic asthma in 8-week female C57BL/6 mice (n=5 per group). B. Shown are total and differential cell counts in BAL cell count. HDM, house dust mite. Treatment groups: Control (DMSO); Low DEX (0.3 mg/kg BW, i.p.); C3G (200 μg i.p./100 μg i.n per mouse); Low DEX/C3G. DEX and C3G were delivered to mice 1 h before HDM challenges. Mice were sacrificed after the last HDM challenge.

FIGS. 4A and 4B provide graphs showing C3G attenuates neutrophilic airway inflammation in a HDM-induced chronic asthma model. A. HDM/CFA-induced chronic asthma in female C57BL/6 mice (sensitization age=8 weeks, n=5 per group). B. Shown are total and differential cell counts in BAL cell count. HDM, house dust mite. Treatment groups: Control (DMSO); Low DEX (0.3 mg/kg BW, i.p.); C3G (200 μg i.p./100 μg i.n per mouse); Low DEX/C3G. DEX and C3G were delivered to mice 24 h before HDM sensitization and 1 h before HDM challenges. Mice were sacrificed after the last HDM challenge.

FIG. 5 provides a graph showing C3G alleviates Th17-mediated experimental autoimmune encephalomyelitis (EAE), which is a model of multiple sclerosis, in mice. MOG33-55-specific Th17 cells were adoptively transferred to 10-week C57BL/6J female mice. The graph shows mean clinical score of EAE after Th17 cell transfer. n=5 per group. *P<0.05 (control vs. low DEX+C3G. Treatment groups: Control (PBS); Low DEX (0.3 mg/kg BW, i.p.); C3G (200 μg i.p. per mouse); Low DEX+C3G. DEX and C3G were delivered to mice every the other day after Th17 transfer.

### DETAILED DESCRIPTION OF THE INVENTION

The inventors have developed a method of treating airway inflammation in a subject by administering a therapeutically effective amount of an anthocyanin 3-0 glucoside and a corticosteroid to a subject in need thereof.

### Definitions

The terminology as set forth herein is for description of the embodiments only and should not be construed as limiting of the invention as a whole. As used in the description of the invention and the appended claims, the singular forms “a”, “an”, and “the” are inclusive of their plural forms, unless contraindicated by the context surrounding such.

As used herein, the term “organic group” is used to mean a hydrocarbon group that is classified as an aliphatic group, cyclic group, or combination of aliphatic and cyclic groups (e.g., alkaryl and aralkyl groups). An alkaryl group is a an aryl group that is attached to the remainder of the structure by an intervening alkyl group, whereas an aralkyl group is an aryl group that is attached directly to the structure but that includes one or more additional alkyl groups attached thereto. In the context of the present invention, suitable organic groups for compounds of this invention are those that do not interfere with the activity of the compounds with regard to treating or preventing airway inflammation. In the context of the present invention, the term “aliphatic group” means a saturated or unsaturated linear or branched hydrocarbon group. This term is used to encompass alkyl, alkenyl, and alkynyl groups, for example.

As used herein, the terms “alkyl”, “alkenyl”, and the prefix “alk-” are inclusive of straight chain groups and branched chain groups. Unless otherwise specified, these groups contain from 1 to 20 carbon atoms, with alkenyl groups containing from 2 to 20 carbon atoms. In some embodiments, these groups have a total of at most 10 carbon atoms, at most 8 carbon atoms, at most 6 carbon atoms, or at most 4 carbon atoms. Alkyl groups including 4 or fewer carbon atoms can also be referred to as lower alkyl groups. Alkyl groups can also be referred to by the number of carbon atoms that they include (i.e., C₁-C₄ alkyl groups are alky groups including 1-4 carbon atoms).

Cycloalkyl, as used herein, refers to an alkyl group (i.e., an alkyl, alkenyl, or alkynyl group) that forms a ring structure. Cyclic groups can be monocyclic or polycyclic and preferably have from 3 to 10 ring carbon atoms. A cycloalkyl group can be attached to the main structure via an alkyl group including 4 or less carbon atoms. Exemplary cyclic groups include cyclopropyl, cyclopropylmethyl, cyclopentyl, cyclohexyl, adamantyl, and substituted and unsubstituted bornyl, norbornyl, and norbornenyl.

Unless otherwise specified, “alkylene” and “alkenylene” are the divalent forms of the “alkyl” and “alkenyl” groups defined above. The terms, “alkylenyl” and “alkenylenyl” are used when “alkylene” and “alkenylene”, respectively, are substituted. For example, an arylalkylenyl group comprises an alkylene moiety to which an aryl group is attached.

The term “haloalkyl” is inclusive of groups that are substituted by one or more halogen atoms, including perfluorinated groups. This is also true of other groups that include the prefix “halo-”. Examples of suitable haloalkyl groups are chloromethyl, trifluoromethyl, and the like. Halo moieties include chlorine, bromine, fluorine, and iodine.

The term “aryl” as used herein includes carbocyclic aromatic rings or ring systems. The aryl groups may include a single aromatic ring, a plurality of separate aromatic rings, or a fused aromatic ring system. Carbocyclic aromatic rings do not include heteroatoms. Examples of aryl groups include phenyl, naphthyl, biphenyl, fluorenyl and indenyl. Aryl groups may be substituted or unsubstituted.

Unless otherwise indicated, the term “heteroatom” refers to the atoms O, S, or N.

The term “heteroaryl” includes aromatic rings or ring systems that contain at least one ring heteroatom (e.g., O, S, N). In some embodiments, the term “heteroaryl” includes a ring or ring system that contains 2 to 12 carbon atoms, 1 to 3 rings, 1 to 4 heteroatoms, and O, S, and/or N as the heteroatoms. Suitable heteroaryl groups include furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, pyrazinyl, 1-oxidopyridyl, pyridazinyl, triazinyl, tetrazinyl, oxadiazolyl, thiadiazolyl, and so on.

The terms “arylene” and “heteroarylene” are the divalent forms of the “aryl” and “heteroaryl” groups defined above. The terms “arylenyl” and “heteroarylenyl” are used when “arylene” and “heteroarylene”, respectively, are substituted. For example, an alkylarylenyl group comprises an arylene moiety to which an alkyl group is attached.

The term “fused aryl group” includes fused carbocyclic aromatic rings or ring systems. Fused aryl groups include a plurality of aromatic rings that are fused to form a single aromatic system. Examples of fused aryl groups include naphthalene (C₁₀), anthracene (C₁₄), phenanthrene (C₁₄) and pyrene (C₁₆) fused aryl groups. Collectively, fused aryl groups can be referred to by reference to the number of carbon ring atoms they contain; i.e., a C₁₀-C₁₈ carboaryl group. The number of rings included in the fused group can be indicated using the terms bicyclic, tricyclic, etc. For example, a bicyclic fused aryl group includes two aryl rings.

The term “fused cycloalkyl aryl group” includes a ring system including both cycloalkyl and aromatic rings that are fused to form a single ring system. A “fused heterocycloalkyl aryl group” is a ring system that includes both a heterocycloalkyl ring and an aromatic ring that are fused to form a single ring system.

When a group is present more than once in any formula or scheme described herein, each group (or substituent) is independently selected, whether explicitly stated or not. For example, for the formula —C(O)—NR₂ each R group is independently selected.

The terms ester, amide, amine, hydroxyl, sulfonate, phosphonate, and guanidine refer to various different functional groups that may be included in compounds of the invention. The functional groups are attached to a carbon atom that forms part of an organic substituent. The functional groups are further described by the following chemical formulas: ester=R—(CO)—O—R; amide=R—(CO)—NH—R; amine=R—NH₂, hydroxyl=R—OH; sulfonate=R—O—SO₃⁻, where R represents the alkyl or aromatic group(s) to which the functional group is attached. Further examples of functional groups are provided below.

Acylamido (acylamino): —NR¹C(═O)R², wherein R¹ is an amide substituent, for example, hydrogen or a C₁₋₇ alkyl group. Examples of acylamide groups include, but are not limited to, —NHC(═O)CH₃, —NHC(═O)CH₂CH₃, and —NHC(═O)Ph. Acylamido groups can be substituted; for example, the acylamido groups can be amine substituted acylamido groups having the formula-NH—CO—(CH₂)ₓ—NH₂, wherein x is an integer from 1-4.

Ureido: —N(R¹)CONR²R³ wherein R² and R³ are independently amino substituents, as defined for amino groups, and R¹ is a ureido substituent, for example, hydrogen or a C₁₋₇ alkyl group. Examples of ureido groups include, but are not limited to, —NHCONH₂, —NHCONHMe, —NHCONHEt, —NHCONMe₂, —NHCONEt₂, —NMeCONH₂, —NMeCONHMe, —NMeCONHEt, —NMeCONMe₂, —NMeCONEt₂ and —NHC(═O)NHPh.

Sulfonyl (sulfone): —S(═O)₂R, wherein R is a sulfone substituent, for example, a C₁₋₇ alkyl group or a C₅₋₂₀ aryl group. Examples of sulfone groups include, but are not limited to, —S(═O)₂CH₃ (methanesulfonyl, mesyl), —S(═O)₂CF₃, —S(═O)₂CH₂CH₃, and 4-methylphenylsulfonyl (tosyl). The sulfone substituent may in some cases be an amino group, as defined above. These groups may be termed “aminosulfonyl” groups.

Sulfonamino: —NR¹S(═O)₂R, wherein R¹ is an amino substituent, as defined for amino groups, and R is a sulfonamino substituent, for example, a C₁₋₇ alkyl group or a C₅₋₂₀ aryl group. Examples of sulfonamino groups include, but are not limited to, —NHS(═O)₂CH₃, —NHS(═O)₂Ph and —N(CH₃)S(═O)₂C₆H₅.

As a means of simplifying the discussion and the recitation of certain terminology used throughout this application, the terms “group” and “moiety” are used to differentiate between chemical species that allow for substitution or that may be substituted and those that do not so allow for substitution or may not be so substituted. Thus, when the term “group” is used to describe a chemical substituent, the described chemical material includes the unsubstituted group and that group with one or more nonperoxidic O, N, S, or F substituents or other conventional substituents such as methyl groups. Where the term “moiety” is used to describe a chemical compound or substituent, only an unsubstituted chemical material is intended to be included. For example, the phrase “alkyl group” is intended to include not only pure open chain saturated hydrocarbon alkyl substituents, such as methyl, ethyl, propyl, tert-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ether groups, haloalkyls, nitroalkyls, carboxyalkyls, hydroxyalkyls, cyanoalkyls, etc. On the other hand, the phrase “alkyl moiety” is limited to the inclusion of only pure open chain saturated hydrocarbon alkyl substituents, such as methyl, ethyl, propyl, tert-butyl, and the like.

The invention is inclusive of the compounds described herein in any of their pharmaceutically acceptable forms, including isomers (e.g., diastereomers and enantiomers), tautomers, salts, solvates, polymorphs, prodrugs, and the like. In particular, if a compound is optically active, the invention specifically includes each of the compound's enantiomers as well as racemic mixtures of the enantiomers. It should be understood that the term “compound” includes any or all of such forms, whether explicitly stated or not (although at times, “salts” are explicitly stated).

Treat“, “treating”, and “treatment”, etc., as used herein, refer to any action providing a benefit to a subject afflicted with airway inflammation or disease such as asthma, including improvement in the condition through lessening or suppression of at least one symptom, delay in progression of the disease, etc.

Prevention, as used herein, refers to any action providing a benefit to a subject at risk of being afflicted with an airway inflammation or disease such as asthma, including avoidance of the development of the condition or disease or a decrease of one or more symptoms of the disease should a disease develop. The subject may be at risk as a result of family history or exposure to irritants.

“Pharmaceutically acceptable” as used herein means that the compound or composition is suitable for administration to a subject for the methods described herein, without unduly deleterious side effects in light of the severity of the disease and necessity of the treatment.

The term “therapeutically effective” is intended to qualify the amount of each agent which will achieve the goal of decreasing disease severity while avoiding adverse side effects such as those typically associated with alternative therapies. The therapeutically effective amount may be administered in one or more doses. An effective dose, on the other hand, is an amount sufficient to provide a certain effect, such as enzyme inhibition, but may or may not be therapeutically effective.

### Anthocyanins

Anthocyanins are glycosides of anthocyanidins, and are water soluble flavonoid pigments that reflect light in the red to blue range of the visible spectrum. Anthocyanins are typically biosynthesized by plants, and have been observed to occur in all tissues of higher plants. The basic chemical structure of anthocyanidin is shown below:

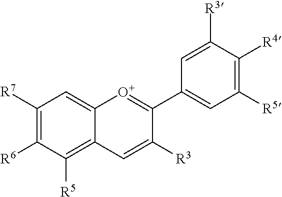

Examples of anthocyanidins include aurantinidin, cyanidin, dephinidin, and europinidin. Anthocyanins are anthocyanidins bearing a sugar group at one of the R positions, most commonly the R³ position, which are referred to as anthocyanin 3-glucosides. Examples of anthocyanins include cyanidin 3-glucoside, delphinidin 3-glucoside, or combinations thereof. In some embodiments, anthocyanins include enantiomer, optical isomer, diastereomer, N-oxide, crystalline form, hydrate, or pharmaceutically acceptable salts of an anthocyanin. The glucose forming the glucoside can be either D- or L-glucose, and bound to the anthocyanidin through a glucose at its 1-position (α-pyranose) via a glucoside bond. Anthocyanins are typically obtained starting from a naturally available precursor, which can be modified if necessary using enzymatic or organic synthesis methods.

In some embodiments, the anthocyanins are anthocyanin 3-glycosides having a formula according to formula I:

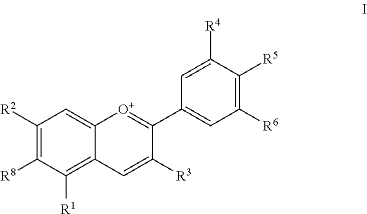

wherein R³ is a glucoside, R⁶, and R⁸ are independently selected from —H, —OH, halogen, —C₁-C₆-alkyl, —C₁-C₆-cycloalkyl, aryl, heteroaryl, —CN, —SO₂—C₁-C₄-alkyl, —SO₂(NH)—C₁-C₄-alkyl, —CF₃, —OCHF₂, —OCF₃, —O—C₁-C₆-alkyl, —O—C₁-C₆-cycloalkyl, —OCH₂CH₂—O—C₁-C₄-alkyl, —SCF₃, —SO₃CF₃, —SF₅, —CONH₂, —CONH—C₁-C₄-alkyl, —CON(C₁-C₄-alkyl)₂; R¹, R², R⁴, and R⁵ are independently selected from —H, —OH, —O—C₁-C₆-alkyl, —O—C₁-C₆-cycloalkyl, halogen, —NH₂, —NH—C₁-C₆-alkyl, —N(C₁-C₆-alkyl)₂, —NH—CO—C₁-C₆-alkyl, —NHSO₂—C₁-C₆-alkyl, —NHSO₂N(C₁-C₆-alkyl)₂, —NHCONH—C₁-C₆-alkyl, —NHCON(C₁-C₆-alkyl)₂, and —NH-aryl; or a pharmaceutically acceptable salt thereof.

In some embodiments, at least one of R¹, R², R⁴, and R⁵ of the compound of formula I is selected from the group consisting of —O—C₁-C₆-alkyl, —O—C₁-C₆-cycloalkyl, halogen, —NH₂, —NH—C₁-C₆-alkyl, —N(C₁-C₆-alkyl)₂, —NH—CO—(C₁-C₆-alkyl), —NHSO₂—C₁-C₆-alkyl, —NHSO₂N(C₁-C₆-alkyl)₂, —NHCONH—C₁-C₆-alkyl, —NHCON(C₁-C₆-alkyl)₂, and —NH-aryl.

In further embodiments, R¹, R², R⁴, and R⁵ are —OH, and R⁶, and R⁸ are independently selected from the group consisting of —H, —C₁-C₄ alkyl, —OH, —OMe, and halogen. In additional embodiments, R⁴ is —OH, while in further embodiments R¹, R², and R⁴ are —OH.

### Treatment of a Steroid-Resistant Disease or Condition Using Anthocyanin and a Corticosteroid

Another aspect of the invention provides a method of treating a steroid-resistant disease or condition in a subject, comprising administering a therapeutically effective amount of an anthocyanin and a corticosteroid to a subject in need thereof. The anthocyanin and corticosteroid can be selected from any of the anthocyanines or corticosteroids described herein.

In some embodiments, the anthocyanin administered to the subject is a compound according to formula I:

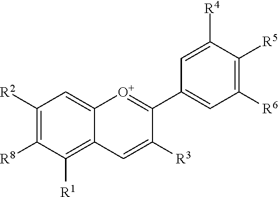

wherein R³ is a glucoside, R⁶, and R⁸ are independently selected from —H, —OH, halogen, —C₁-C₆-alkyl, —C₁-C₆-cycloalkyl, aryl, heteroaryl, —CN, —SO₂—C₁-C₄-alkyl, —SO₂(NH)—C₁-C₄-alkyl, —CF₃, —OCHF₂, —OCF₃, —O—C₁-C₆-alkyl, —O—C₁-C₆-cycloalkyl, —OCH₂CH₂—O—C₁-C₄-alkyl, —SCF₃, —SO₃CF₃, —SF₅, —CONH₂, —CONH—C₁-C₄-alkyl, —CONH(C₁-C₄-alkyl)₂; R¹, R², R⁴, and R⁵ are independently selected from —H, —OH, —O—C₁-C₆-alkyl, —O—C₁-C₆-cycloalkyl, halogen, —NH₂, —NH—C₁-C₆-alkyl, —NH(C₁-C₆-alkyl)₂, —NH—CO—C₁-C₆-alkyl, —NHSO₂—C₁-C₆-alkyl, —NHSO₂N(C₁-C₆-alkyl)₂, —NHCONH—C₁-C₆-alkyl, —NHCON(C₁-C₆-alkyl)₂, —NH-aryl; or a pharmaceutically acceptable salt thereof.

The anthocyanin and corticosteroid can be used to provide prophylactic and/or therapeutic treatment to a subject. A subject in need thereof, as defined herein, is a subject who has been diagnosed as having a steroid-resistant disease or condition, or has an increased risk of developing a disease characterized by steroid-resistance. The compounds of the invention can, for example, be administered prophylactically to a subject in advance of the occurrence of airway inflammation or a disease characterized by airway inflammation. Prophylactic (i.e., preventive) administration is effective to decrease the likelihood of the subsequent occurrence of a steroid-resistant disease or condition in the subject, or decrease the severity of the steroid-resistant disease or condition that subsequently occurs. Prophylactic treatment may be provided to a subject that is at elevated risk of developing a steroid-resistant disease or condition, such as a subject with a family history of asthma or exposure to known risk factors for airway inflammation.

Alternatively, the anthocyanin and corticosteroid can be administered therapeutically to a subject that is already afflicted by a steroid-resistant disease or condition. In one embodiment of therapeutic administration, administration of the compounds is effective to eliminate the steroid-resistant disease or condition; in another embodiment, administration of the compounds is effective to decrease the severity of the steroid-resistant disease or condition or to lengthen the lifespan of the subject so afflicted. The subject is preferably a mammal, such as a domesticated farm animal (e.g., cow, horse, pig) or pet (e.g., dog, cat). More preferably, the subject is a human.

The present invention involves administering a therapeutically effective amount of an anthocyanin and a corticosteroid to a subject. Corticosteroids are a class of steroid hormones that are produced in the adrenal cortex of vertebrates, as well as the synthetic analogues of these hormones. Two main classes of corticosteroids are glucocorticoids and mineralocorticoids. Examples of corticosteroids include cortisol, cortisone, prednisone, prednisolone, methylprednisolone, dexamethasone, betamethasone, fluticasone, budesonide, mometasone, beclometasone, triamcinolone, fludrocortisone acetate, and deoxycorticosterone acetate. A preferred corticosteroid for use in the present method is dexamethasone.

The method of the invention involves co-administration of an anthocyanin and a corticosteroid. Co-administration refers to the administration of two or more therapeutic agents to treat airway inflammation or a disease characterized by airway inflammation. Such administration encompasses administration of these therapeutic agents in a substantially simultaneous manner, such as in a single dose having a fixed ratio of active ingredients or in multiple, separate doses for each active ingredient. In addition, such administration also encompasses use of anthocyanin and a corticosteroid in a sequential manner. In either case, the treatment regimen will provide beneficial effects of the drug combination in treating airway inflammation or a disease characterized by airway inflammation.

The term “steroid-resistant disease or condition” refers to a disease or condition that shows an insufficient clinical response to administered steroids, wherein the condition is typically treated with such steroids. Non-limiting examples of disorders to be treated herein include conditions and diseases such as multiple sclerosis, systemic lupus erythematosus, and inflammatory bowel disease, and conditions and diseases of the airways, including, but not limited to, airway inflammation, airway hyper-responsiveness, asthma, and chronic obstructive pulmonary disease.

Subjects in which administration of a steroid, typically effective in patients having such diseases and healthy persons, is ineffective, are considered “steroid resistant” or “steroid refractory” subjects, which in the present invention include also subjects who respond poorly or inadequately, i.e. exhibit only a slight effect or improvement of symptoms. The term “steroid resistant” includes both acquired steroid resistance (Type I) and primary steroid resistance (Type II).

The determination of whether a patient is steroid refractory is difficult as no universally accepted method or indeed end-point exists which can be used to assess such a condition. Moreover, steroid unresponsiveness is a dynamic scale and there are varying degrees of unresponsiveness. From a clinical perspective, the diagnosis of steroid unresponsiveness is often based on the physician's judgment and treatment history with steroids. For example, in steroid-resistant asthma, increasing the dose of steroids for a certain period of time without achieving symptomatic relief may be indicative of a possible state of steroid unresponsiveness in that individual.

The method of the invention is used to treat or prevent the development of airway inflammation or a disease characterized by airway inflammation in a subject. Airway inflammation is a state of irritation in the airways where the immune system responds to a perceived threat and causes swelling, itching, redness, and other symptoms. Airway inflammation can be either acute or chronic airway inflammation. Diseases characterized by airway inflammation are known to those skilled in the art, and include asthma, chronic bronchitis, bronchiectasis, and cystic fibrosis. See Shelhamer et al., Ann Intern Med. 123(4), 288-304 (1995).

In some embodiments, the subject has been diagnosed as having asthma. Asthma is a long-term inflammatory disease of the airways of the lungs. Symptoms of asthma include episodes of wheezing, coughing, chest tightness, and shortness of breath. While asthma is a well-recognized condition, there is currently no precise test for the diagnosis, which is typically based on the pattern of symptoms and response to therapy over time. A diagnosis of asthma should be suspected if there is a history of recurrent wheezing, coughing or difficulty breathing and these symptoms occur or worsen due to exercise, viral infections, allergens or air pollution. The best method for diagnosing asthma is spirometry, which is a test carried out using a spirometer that measures lung function, specifically the amount (volume) and/or speed (flow) of air that can be inhaled and exhaled

In some embodiments, the subject has been diagnosed as having severe asthma. Severe asthma, also known as acute severe asthma or status asthmaticus, is an acute exacerbation of asthma that does not respond to standard treatments of bronchodilators (inhalers) and corticosteroids. Symptoms include chest tightness, rapidly progressive dyspnea (shortness of breath), dry cough, use of accessory respiratory muscles, fast and/or labored breathing, and extreme wheezing. In severe asthma, breathlessness may be so severe that it is impossible to speak more than a few words (inability to complete sentences. Severe asthma is a life-threatening episode of airway obstruction and is considered a medical emergency. Severe asthma is characterized histologically by smooth muscle hypertrophy and basement membrane thickening.

In some embodiments, the asthma is steroid-resistant asthma. Steroid-resistant asthma refers to inflammation and constriction of the airways that does not respond well to treatment with steroids. However, higher than normal doses of steroids will alleviate symptoms in most patients having steroid-resistant asthma, but the higher doses required carry an increased risk of side effects from steroid use.

In some embodiments, the steroid-resistant disease or condition is COPD. The term “chronic obstructive pulmonary disease” or “COPD” refers to a progressive disease characterized by difficulty breathing, coughing that produces a large amount of mucus, wheezing, shortness of breath, and/or chest tightness. COPD is typically caused by cigarette smoking and/or long-term exposure to other lung irritants, such as air pollution, chemical fumes, or dust. COPD includes both emphysema and chronic bronchitis. COPD is typically steroid-resistant.

In some embodiments, the steroid-resistant disease or condition is SLE. The term “systemic lupus erythematosus” (“lupus” or “SLE”) refers to an autoimmune disorder in which a patient's immune system produces auto-antibodies, causing widespread inflammation and tissue damage. SLE can affect many systems and tissues, including joints, skin, brain, lungs, kidneys, and blood vessels, and patients with SLE may experience fatigue, pain, swelling in their joints, skin rashes, and fevers. In some embodiments, SLE is steroid-resistant.

In some embodiments, the steroid-resistant disease or condition is IBD. The term “inflammatory bowel disease” (“IBD”) refers to a group of chronic intestinal diseases characterized by inflammation of the bowel (both the large and small intestine). Nonlimiting exemplary inflammatory bowel diseases include ulcerative colitis, characterized by inflammation of the mucosa (inner lining) of the intestine, and Crohn's disease, characterized by inflammation throughout the bowel wall. While IBD may be limited to the intestine, it can also affect the skin, joints, spine, liver, eyes, and other organs. In some embodiments, IBD is steroid-resistant.

Candidate agents may be tested in animal models. The animal model should be one appropriate for a steroid-resistant disease or condition. For example, the animal model can be one for the study of asthma, and in particular steroid-resistant asthma. The study of airway inflammation in animal models (for instance, mice) is a commonly accepted practice for evaluating agents for their ability to treat airway inflammation. For instance, the house dust mite (HDM)/complete Freund's adjuvant (CFA)-induced neutrophilic mouse model can be used to evaluate agents for their ability to treat steroid-resistant asthma. Results are typically compared between control animals treated with candidate agents and the control littermates that did not receive treatment. Candidate agents can be used in these animal models to determine if a candidate agent decreases one or more of the symptoms associated with a disease characterized by airway inflammation, including, for instance, inflammation, cytokine levels, or combinations thereof.

### Administration and Formulation

The present invention also provides pharmaceutical compositions that include an anthocyanin and a corticosteroid as the active ingredients, and a pharmaceutically acceptable liquid or solid carrier or carriers, in combination with the active ingredients. Either or both of the active ingredients can be delivered together with a pharmaceutically acceptable carrier, and can be delivered using the same or different pharmaceutically acceptable carriers. Any of the compounds described above as being suitable for the treatment of airway inflammation and diseases characterized by airway inflammation can be included in pharmaceutical compositions of the invention.

The compounds can be administered as pharmaceutically acceptable salts. Pharmaceutically acceptable salt refers to the relatively non-toxic, inorganic and organic acid addition salts of the compounds. These salts can be prepared in situ during the final isolation and purification of the compound, or by separately reacting a purified compound according to formula I with a suitable counterion, depending on the nature of the compound, and isolating the salt thus formed. Representative counterions include the chloride, bromide, nitrate, ammonium, sulfate, tosylate, phosphate, tartrate, ethylenediamine, and maleate salts, and the like. See for example Haynes et al., J. Pharm. Sci., 94, p. 2111-2120 (2005).

The pharmaceutical compositions include an anthocyanin and a corticosteroid together with one or more of a variety of pharmaceutically acceptable carriers for delivery to a subject, including a variety of diluents or excipients known to those of ordinary skill in the art. “Pharmaceutically acceptable carrier” refers herein to a composition suitable for delivering an active pharmaceutical ingredient, such as the composition of the present invention, to a subject without excessive toxicity or other complications while maintaining the biological activity of the active pharmaceutical ingredient. For example, for parenteral administration, isotonic saline is preferred. For topical administration, a cream, including a carrier such as dimethylsulfoxide (DMSO), or other agents typically found in topical creams that do not block or inhibit activity of the peptide, can be used. Other suitable carriers include, but are not limited to, alcohol, phosphate buffered saline, and other balanced salt solutions.

The formulations may be conveniently presented in unit dosage form and may be prepared by any of the methods well known in the art of pharmacy. Preferably, such methods include the step of bringing the active agent into association with a carrier that constitutes one or more accessory ingredients. In general, the formulations are prepared by uniformly and intimately bringing the active agent into association with a liquid carrier, a finely divided solid carrier, or both, and then, if necessary, shaping the product into the desired formulations. The methods of the invention include administering to a subject, preferably a mammal, and more preferably a human, the composition of the invention in an amount effective to produce the desired effect. The anthocyanin and corticosteroid can be administered as a single dose or in multiple doses. Useful dosages of the active agents can be determined by comparing their in vitro activity and their in vivo activity in animal models. Methods for extrapolation of effective dosages in mice, and other animals, to humans are known in the art; for example, see U.S. Pat. No. 4,938,949.

The agents of the present invention are preferably formulated in pharmaceutical compositions and then, in accordance with the methods of the invention, administered to a subject, such as a human patient, in a variety of forms adapted to the chosen route of administration. The formulations include, but are not limited to, those suitable for oral, inhaled, rectal, vaginal, topical, nasal, ophthalmic, or parenteral (including subcutaneous, intramuscular, intraperitoneal, and intravenous) administration.

Formulations of the present invention suitable for oral administration may be presented as discrete units such as tablets, troches, capsules, lozenges, wafers, or cachets, each containing a predetermined amount of the active agent as a powder or granules, as liposomes containing the active compound, or as a solution or suspension in an aqueous liquor or non-aqueous liquid such as a syrup, an elixir, an emulsion, or a draught. Such compositions and preparations typically contain at least about 0.1 wt-% of the active agent. The amount of the compound according to formula I (i.e., active agent) is such that the dosage level will be effective to produce the desired result in the subject.

Inhaled formulations are preferred for the treatment of airway inflammation and diseases characterized by airway inflammation. Inhaled formulations include those designed for administration from an inhaler device. Compositions for inhalation or insufflation include solutions and suspensions in pharmaceutically acceptable, aqueous or organic solvents, or mixtures thereof, aerosols, and powders. Preferably, the compositions are administered by the oral or nasal respiratory route for local or systemic effect. Compositions in preferably pharmaceutically acceptable solvents may be nebulized by use of inert gases. Solution, suspension, or powder compositions may be administered, preferably orally or nasally, from devices that deliver the formulation in an appropriate manner. In some embodiments, the anthocyanin and/or the corticosteroid are administered as an aerosol formulation. Nasal spray formulations include purified aqueous solutions of the active agent with preservative agents and isotonic agents. Such formulations are preferably adjusted to a pH and isotonic state compatible with the nasal mucous membranes.

Formulations for rectal or vaginal administration may be presented as a suppository with a suitable carrier such as cocoa butter, or hydrogenated fats or hydrogenated fatty carboxylic acids. Ophthalmic formulations are prepared by a similar method to the nasal spray, except that the pH and isotonic factors are preferably adjusted to match that of the eye. Topical formulations include the active agent dissolved or suspended in one or more media such as mineral oil, petroleum, polyhydroxy alcohols, or other bases used for topical pharmaceutical formulations.

The tablets, troches, pills, capsules, and the like may also contain one or more of the following: a binder such as gum tragacanth, acacia, corn starch or gelatin; an excipient such as dicalcium phosphate; a disintegrating agent such as corn starch, potato starch, alginic acid, and the like; a lubricant such as magnesium stearate; a sweetening agent such as sucrose, fructose, lactose, or aspartame; and a natural or artificial flavoring agent. When the unit dosage form is a capsule, it may further contain a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, sugar, and the like. A syrup or elixir may contain one or more of a sweetening agent, a preservative such as methyl- or propylparaben, an agent to retard crystallization of the sugar, an agent to increase the solubility of any other ingredient, such as a polyhydric alcohol, for example glycerol or sorbitol, a dye, and flavoring agent. The material used in preparing any unit dosage form is substantially nontoxic in the amounts employed. The active agent may be incorporated into sustained-release preparations and devices.

### Preparation of the Compounds

Compounds of the invention may be synthesized by synthetic routes that include processes similar to those well known in the chemical arts, particularly in light of the description contained herein. The starting materials are generally available from commercial sources such as Aldrich Chemicals (Milwaukee, Wis., USA) or are readily prepared using methods well known to those skilled in the art (e.g., prepared by methods generally described in Louis F. Fieser and Mary Fieser, Reagents for Organic Synthesis, v. 1-19, Wiley, New York, (1967-1999 ed.); Alan R. Katritsky, Otto Meth-Cohn, Charles W. Rees, Comprehensive Organic Functional Group Transformations, v 1-6, Pergamon Press, Oxford, England, (1995); Barry M. Trost and Ian Fleming, Comprehensive Organic Synthesis, v. 1-8, Pergamon Press, Oxford, England, (1991); or Beilsteins Handbuch der organischen Chemie, 4, Aufl. Ed. Springer-Verlag, Berlin, Germany, including supplements (also available via the Beilstein online database)).

Those skilled in the art will appreciate that various synthetic routes may be used to synthesize the compounds of the invention. Although specific starting materials and reagents are depicted in the reaction schemes and discussed below, other starting materials and reagents can be easily substituted to provide a variety of derivatives and/or reaction conditions. In addition, many of the compounds prepared by the methods described below can be further modified in light of this disclosure using conventional methods well known to those skilled in the art.

The present invention is illustrated by the following example. It is to be understood that the particular example, materials, amounts, and procedures are to be interpreted broadly in accordance with the scope and spirit of the invention as set forth herein.

### EXAMPLE

### Effects of Cyanidin-3-O-Glucoside (C3G) on Airway Inflammation

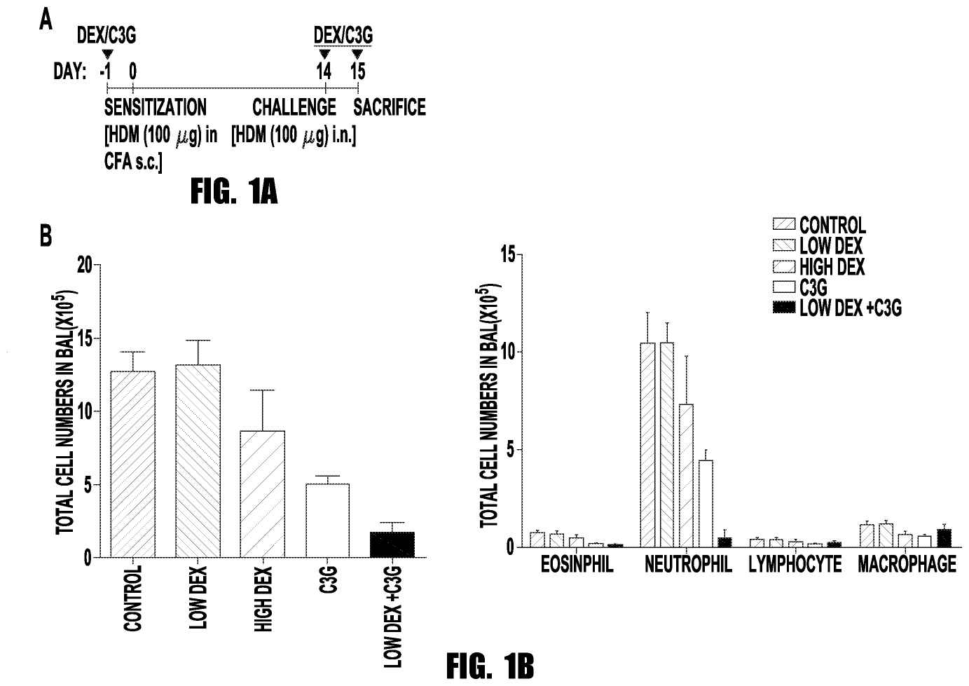

C3G attenuates neutrophilic airway inflammation in a HDM-induced acute asthma model. House dust mite (HDM), a natural allergen to which asthmatics are often sensitized, can induce neutrophilic airway inflammation, which is often associated with steroid-resistant severe asthma. The inventors have established an HDM/CFA-induced neutrophilic severe asthma model. The ability of C3G in combination with low-dose DEX (low DEX) was evaluated in alleviating neutrophilic airway inflammation in a HDM-induced steroid-resistant severe asthma model. Eight-week WT C57BL/6 female mice were sensitized subcutaneously (s.c.) with HDM (100 μg per mouse) in Complete Freund's Adjuvant (CFA) on day 0 and subsequently challenged (i.n.) with HDM (100 μg per mouse) on day 14. BAL cell counting and tissue collection were performed after 24 h of last HDM challenge. Low DEX (0.3 mg/kg BW), high-dose DEX (high DEX. 3 mg/kg BW), C3G (120 μg i.p./60 μg i.n per mouse), or low DEX/C3G combination were administrated into the mice on day 14 (24 h before HDM sensitization and 1 h before HDM challenge). As shown in FIG. 1, HDM induced strong neutrophilic airway inflammation as indicated by bronchoalvelar lavage (BAL) cell count and inflammatory gene expression in the lung (FIG. 1 A-B). Low DEX did not show much effect on inflammation alleviation, validating this is a steroid-resistant asthma model. Although airway inflammation was reduced to some degree by high DEX and C3G treatment alone, a dramatic reduction in inflammation was observed by using low DEX/C3G combination. The above results from indicate that low DEX/C3G combination is a novel and effective method treating steroid-resistant severe asthma.

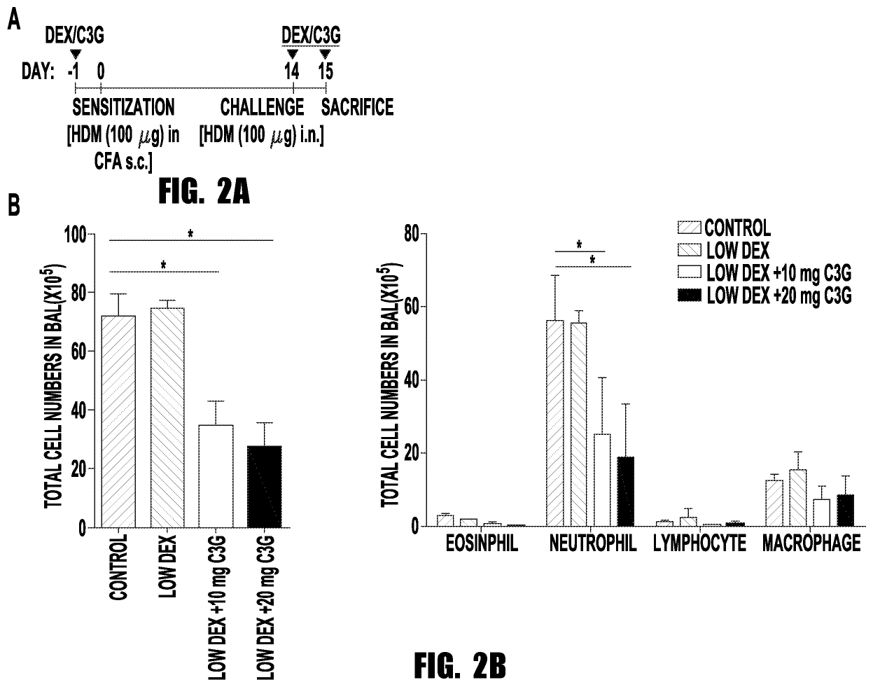

Oral C3G attenuates neutrophilic airway inflammation in a HDM-induced acute asthma model. The inventors also examined if C3G can attenuate neutrophilic airway inflammation in a HDM-induced acute asthma upon oral administration. As shown in FIG. 2, in the presence of low DEX, oral delivery of C3G (by gavage) at a dose of 10 mg/mouse was able to inhibit airway inflammation as indicated by BAL cell count ((FIGS. 2A and 2B). Increase of C3G dose (20 mg/mouse) did not improve the efficacy further. The results indicate that oral C3G has more than 50 times lower bioavailability than C3G delivered by i.p or i.n. (also see FIG. 1)

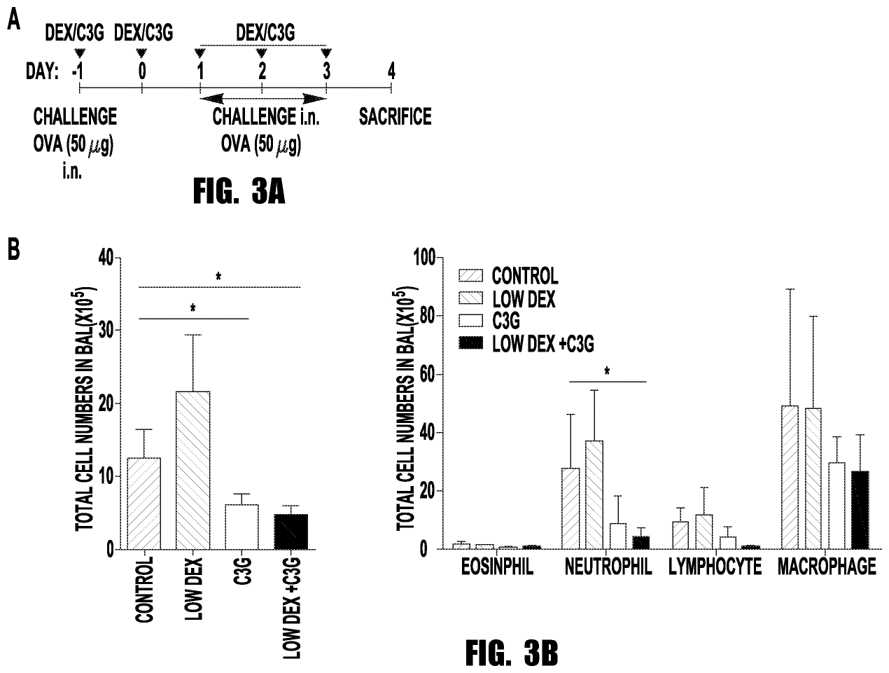

C3G attenuates neutrophilic airway inflammation in a Th17-induced asthma model. To further confirm the synergistic effect of DEX/C3G in IL-17A-driven asthma, the efficacy of C3G was tested in a TH17 cell-mediated neutrophilic asthma model (FIG. 3). Indeed, although low DEX had trend to worsen neutrophilic airway inflammation, C3G alone or in combination with low DEX alleviated the inflammation dramatically.

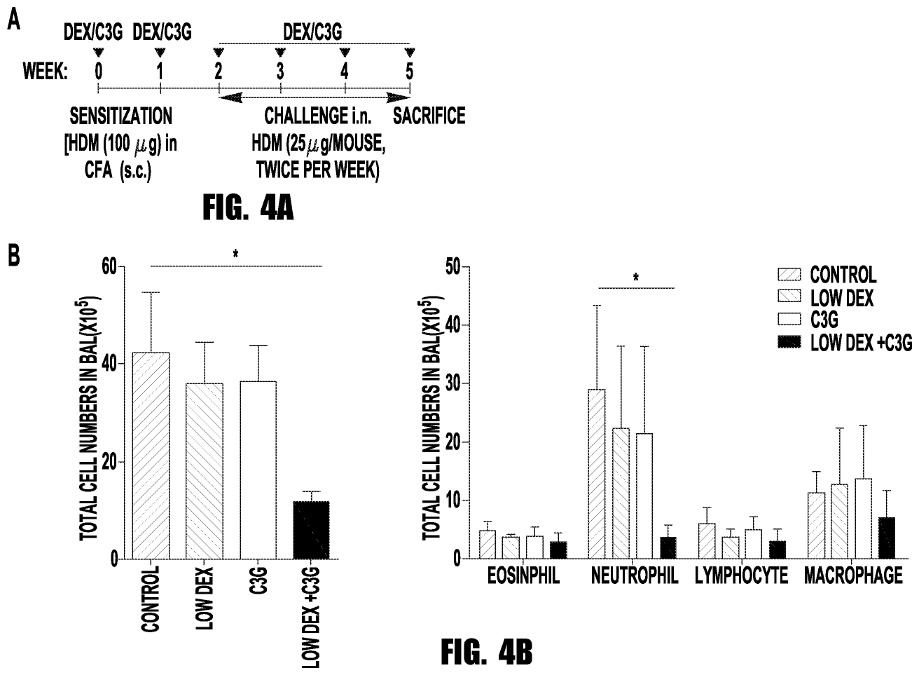

C3G attenuates neutrophilic airway inflammation in a HDM-induced chronic asthma model. Asthma is chronic inflammation disease in human. The inventors thus also investigated the efficacy of C3G in a neutrophilic chronic asthma model. As shown in FIG. 4, the inventors found that C3G in combination with low DEX was very effective in inhibiting neutrophilic inflammation in chronic asthma.

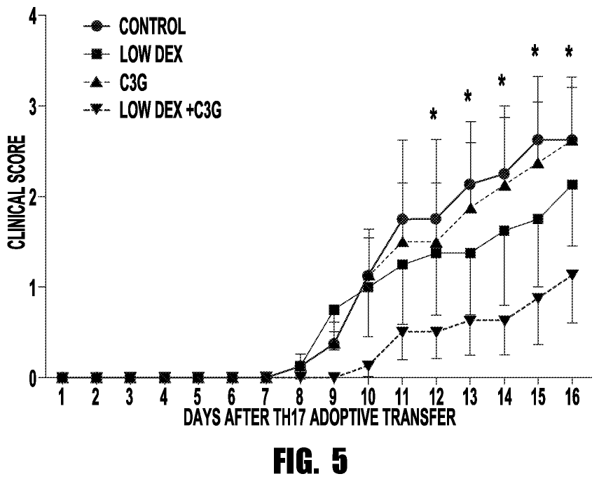

C3G alleviates Th17-mediated EAE in mice. IL-17A-producing Th17 cells play a critical role in the development and pathogenesis of experimental autoimmune encephalomyelitis (EAE). EAE is markedly suppressed in mice lacking IL-17A or IL-17A receptor (IL-17ARA and IL-17ARC). Therefore, Th17-mediated EAE is a suitable animal disease model to test the efficacy of C3G. As shown in FIG. 5, C3G in combination with low DEX substantially delayed onset of EAE and attenuated disease severity

The complete disclosure of all patents, patent applications, and publications, and electronically available materials cited herein are incorporated by reference. The foregoing detailed description and examples have been given for clarity of understanding only. No unnecessary limitations are to be understood therefrom. In particular, while theories may be presented describing possible mechanisms through with the compounds are effective, the inventors are not bound by theories described herein. The invention is not limited to the exact details shown and described, for variations obvious to one skilled in the art will be included within the invention defined by the claims.

1. A method of treating a steroid-resistant disease or condition in a subject, comprising administering a therapeutically effective amount of an anthocyanin and a corticosteroid to a subject in need thereof.

2. The method of claim 1, wherein the anthocyanin is a 3-0 glucoside anthocyanin.

3. The method of claim 1, wherein the anthocyanin is a compound according to formula I 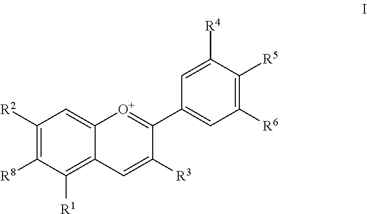
wherein R³ is a glucoside, R⁶, and R⁸ are independently selected from —H, —OH, halogen, —C₁-C₆-alkyl, —C₁-C₆-cycloalkyl, aryl, heteroaryl, —CN, —SO₂—C₁-C₄-alkyl, —SO₂(NH)—C₁-C₄-alkyl, —CF₃, —OCHF₂, —OCF₃, —O—C₁-C₆-alkyl, —O—C₁-C₆-cycloalkyl, —OCH₂CH₂—O—C₁-C₄-alkyl, —SCF₃, —SO₃CF₃, —SF₅, —CONH₂, —CONH—C₁-C₄-alkyl, —CONH(C₁-C₄-alkyl)₂; R¹, R², R⁴, and R⁵ are independently selected from —H, —OH, —O—C₁-C₆-alkyl, —O—C₁-C₆-cycloalkyl, halogen, —NH₂, —NH—C₁-C₆-alkyl, —NH(C₁-C₆-alkyl)₂, —NH—CO—C₁-C₆-alkyl, —NHSO₂—C₁-C₆-alkyl, —NHSO₂N(C₁-C₆-alkyl)₂, —NHCONH—C₁-C₆-alkyl, —NHCON(C₁-C₆-alkyl)₂, —NH-aryl; or a pharmaceutically acceptable salt thereof.

4. The method of claim 3, wherein R¹, R², R⁴, and R⁵ are —OH, and R⁶, and R⁸ are independently selected from the group consisting of —H, —C₁-C₄ alkyl, —OH, —OMe, and halogen.

5. The method of claim 3, wherein R⁴ is —OH.

6. The method of claim 3, wherein R¹, R², and R⁴ are —OH.

7. The method of claim 1, wherein the anthocyanin is cyanidin-3-glucoside.

8. The method of claim 1, wherein the steroid-resistant disease or condition is airway inflammation.

9. The method of claim 8, wherein the subject has been diagnosed as having asthma.

10. The method of claim 9, wherein the subject has been diagnosed as having severe asthma.

11. The method of claim 9, wherein the asthma is steroid-resistant asthma.

12. The method of claim 1, wherein the steroid-resistant disease or condition is steroid-resistant multiple sclerosis.

13. The method of claim 1, wherein the compounds are administered with a pharmaceutically acceptable carrier.

14. The method of claim 1, wherein the corticosteroid is dexamethasone

15. The method of claim 1, wherein the anthocyanin is administered as an aerosol formulation.

16. The method of claim 1, wherein the anthocyanin and corticosteroid are administered substantially simultaneously.
